(19) United States
(12) Patent Application Publication
(10) Pub. No.: US 2007/0123760 A1
(43) Pub. Date: May 31, 2007

(54) Signal transmitter and control circuit for a physiological variable

(76) Inventors: Bernd Scholler, Karlsruhe (DE); Thomas Marx, Hamburg (DE); Elmar Vitt, Rotenburg (DE); Thomas Magin, Schifferstadt (DE); Klaus Forstner, Asperg (DE)

Correspondence Address: Friedrich Kueffner, Suite 910, 317 Madison Avenue, New York, NY, 10017, US

(21) Appl. No.: 11/600,333

(22) Filed: Nov. 15, 2006

(30) Foreign Application Priority Data
Nov. 15, 2005 (DE) 10 2005 054 837.7

(51) Int. Cl. A61B 5/00

(52) U.S. Cl. 600/310

(57) ABSTRACT

For detecting and processing a physiological variable, a signal transmitter has devices for processing, using, and/or providing signals, which are generated from measurement values of the physiological variable. In accordance with a sequence control approach, the measurement values are acquired from the detection of electromagnetic waves of different wavelengths. Before the signals are detected, the electromagnetic waves pass through the medium to be examined or are reflected by this medium. For at least a certain percentage of the overall signal quantity, at least two measurement values detected close together in time are used for each generated signal. The signal transmitter is suitable for use in a control circuit, which is designed with an actuator to influence the physiological variable detected instrumentally by the signal transmitter.

### BACKGROUND OF THE INVENTION

### 1. Field of the Invention

The present invention relates to a signal transmitter, which determines a physiological variable, where the variable can be the temperature, the heart rate, the pH value, or the concentration of hemoglobin (cHb), of oxyhemoglobin (HbO₂), of deoxygenated hemoglobin (HbDe), of carboxyhemoglobin (HbCO), of methemoglobin (MetHb), of sulfhemoglobin (HbSulf), of bilirubin, of glucose, of bile pigments, or SaO₂, SaCO, SpO₂, CaO₂, SpCO, etc., and to the use of the physiological variable for the manual and automatic control of additional internal or external signal receivers such as therapeutic dialysis devices, perfusors, infusors, ventilators, etc.

The invention also relates to a control circuit consisting of a signal transmitter and a signal receiver for the control of a physiological variable.

In particular, the invention relates to a signal transmitter which noninvasively records, compensates, and processes a physiological variable (pV) to provide an output signal, which represents the value of the pV at the time of the measurement.

There is a need for the ability to determine noninvasively physiological variables (pV) with high accuracy and speed, so that this information can be used to drive signal receivers such as therapeutic devices, which, in the manner of an actuator, in turn influence the pV either directly or indirectly or maintain it in a predetermined range. As a result, it becomes possible to construct a control circuit, to which one or more actuating variables are transmitted such as cHb, SpO₂, CaO₂, etc. The variable pV is compared by way of feedback with the actuating variable, and an actuator is driven correspondingly by a controller. The source of electromagnetic radiation is, for example, one or more LEDs and/or one or more laser diodes.

The electromagnetic radiation is selected from one or more ranges of 150 nm±15%, 400 nm±15%, 460 nm±15%, 480 nm±15%, 520 nm±15%, 550 nm±15%, 560 nm±15%, 606 nm±15%, 617 nm±15%, 620 nm±15%, 630 nm±15%, 650 nm±15%, 660 nm±, 705 nm±15%, 710 nm±15%, 720 nm±10%, 805 nm±15%, 810 nm ±15%, 880 nm±15%, 905 nm±15%, 910 nm±15%, 950 nm±15%, 980 nm±15%, 980 nm±15%, 1050 nm±15%, 1200 nm±15%, 1310 nm±15%, 1380 nm±15%, 1450 nm±15%, 1600 nm±15%, 1800 nm±15%, 2100 nm±15%, 2800 nm±15%.

The electromagnetic waves are conducted through a living and/or dead medium to be studied, preferably animal and/or human tissue.

The transmitted and/or the reflected component of the electromagnetic waves is detected by one or more receiving systems. The receiving system is able to detect various wavelengths. The receiving system is also able to record the detected electromagnetic waves and/or to store and/or to transmit them, such as in the form of at least one electric pulse.

The minimum of one signal is processed by an evaluation unit by means of signal conditioning. Independently of the original wavelength, the minimum of one signal is subjected to further processing by means of active and/or passive electronic components. It is preferable for the signal to be adapted with respect to frequency and amplitude.

Digital signals which are representative of at least two different wavelengths of the originally emitted electromagnetic radiation are analyzed by at least one CPU. For this purpose it is preferable to provide an analyzer in the area of the CPU. Signal processing is preferably carried out in the area of the CPU. At least one data storage unit for the digital signals, from which the data can be retrieved, is preferably provided in the area of the CPU.

In the area of the analyzer, the following operations are carried out alternatively, sequentially, or simultaneously:

measurement values are acquired and processed;

a pulse wave characteristic or morphology or parameters derived therefrom such as extremes, derivatives, etc., are obtained;

extinctions are determined (calculated or read out);

internal and external artifacts are cleaned up (motion, repositioning, perfusion;

parallel series of measurements are back-calculated and combined to obtain a new result;

an analog or digital signal is calculated and conditioned to control additional modules or devices.

As a result, the CPU supplies data which are representative of at least one pV of the exposed medium.

Artifacts are preferably cleaned up by a CPU, which processes the output signal of the evaluation unit in the time domain (e.g., a polynomial function) or in the Laplace domain (e.g., by means of a Fourier transformation or wavelets). The functions are selected in such a way that they are adapted to the properties of the possible artifacts.

By the use of a compensation method, the pVs are typically determined with an accuracy of at least 5%, and preferably of 2%, over the measuring range of the pV in question.

For example, the measuring range for the concentration of hemoglobin cHb is typically 5-20 g/dL, where normal values are 14-18 g/dL for men and 12-16 g/dL for women.

### SUMMARY OF THE INVENTION

In general, the compensatory mechanisms of the body are operating at full capacity at Hkt 24%/cHb 8 g/dL even at normal O₂ consumption levels and under otherwise favorable conditions.

According to the invention, it is therefore proposed that cHb be determined noninvasively with an accuracy of 1.5-2.0 g/dL, and preferably of 1 g/dL, where the measurement value for cHb can be made available preferably in less than 10 seconds.

The value range for bilirubin is typically 0.1-5.0 mg/dL.

According to the invention, it is therefore proposed that bilirubin be determined noninvasively with an accuracy of 0.1-1.0 mg/dL, and preferably of 0.5 mg/dL, where the measurement value for bilirubin can be made available preferably in less than 10 seconds.

The value range for blood oxygen O₂ is typically 50-100% saturation, where physiological and/or pathophysiological fluctuations can exert their effects very quickly.

According to the invention, it is therefore proposed that oxygen be determined noninvasively with an accuracy of 5%, and preferably of 2%, where the measurement value for oxygen can be made available in less than 60 seconds, and preferably in less than 10 seconds.

According to the invention, it is therefore proposed that methemoglobin be determined noninvasively with an accuracy of 5%, and preferably of 2%, where the measurement value for oxygen can be made available in less than 60 seconds, preferably in less than 10 seconds.

According to the invention, it is therefore proposed that carboxyhemoglobin (SaCO) be determined noninvasively with an accuracy of 5%, and preferably of 2%, where the measurement value for oxygen can be made available in less than 60 seconds, preferably in less than 10 seconds.

The accuracy and speed of the determination of the pV depends on the available electrical energy and the time required to calculate the measurement signals. Especially in the case of transportable devices which are operated on battery power, the amount of energy available is a limiting factor.

Errors in the operating behavior of the signal transmitter can be complex and can be a nonlinear function of many variables. The pV contributes directly to the error, whereas secondary process variables (which influence the measurement of the primary process variables) enter indirectly into the error. Because the demand for accuracy is increasing, the contributions of the secondary variables are becoming more important.

In addition to the problems of software complexity and calculation complexity, the energy consumption of the CPU of the signal transmitter, for example, is critical, because all of the operating energy or supply voltage flows through the same lines as those used for communications. In addition, several intrinsically safe areas limit the energy available to the signal transmitter. The limited current supply limits not only the number and complexity of the calculations but also the functionality which can be realized in the signal transmitter.

Especially the sensor, the CPU, and signal processing require a great deal of energy.

There is therefore a need for an accurate method for automatically controlling pVs which is simple in terms of calculation and which requires only a small number of stored property constants, so that the amount of energy consumed is reduced, an increased amount of energy is available for additional functionality, and increased updating speeds or signal transmission rates are available and/or a faster CPU can be provided.

According to the invention, it is therefore proposed that all functions of the signal transmitter be combined on preferably two or fewer circuit boards in order to minimize the energy demand. It is also proposed according to the invention that a black-and-white display and/or a gray-scale display be used instead of a color display.

According to the invention, it is proposed that, to increase the speed, the pulse wave be identified quickly by presetting the A/D converter to the bandwidth of the expected pulsation signal.

According to the invention, it is also proposed that, as another way of increasing the speed, a 24-bit A/D converter be used for the further processing of the signal received from the PD.

It is therefore possible according to the invention to identify the pulse wave in no more than 20 msec.

According to the invention, it is also proposed that, as another way of increasing the speed, the transmission of the electromagnetic wavelengths be used as an automatic control parameter.

The various features of novelty, which characterize the invention, are pointed out with particularity in the claims annexed to and forming part of the disclosure. For a better understanding of the invention, its operating advantages, and specific objects attained by its use, reference should be had to the drawing and descriptive matter in which there are illustrated and described preferred embodiments of the invention.

### BRIEF DESCRIPTION OF THE DRAWING

In the drawing:

FIG. 1 shows a schematic diagram of the circuit of the signal transmitter;

FIG. 2 shows a schematic diagram of a control circuit consisting of the signal transmitter, a signal receiver, and a human being; and

FIG. 3 shows a schematic diagram of the signal transmitter.

### DETAILED DESCRIPTION OF THE INVENTION

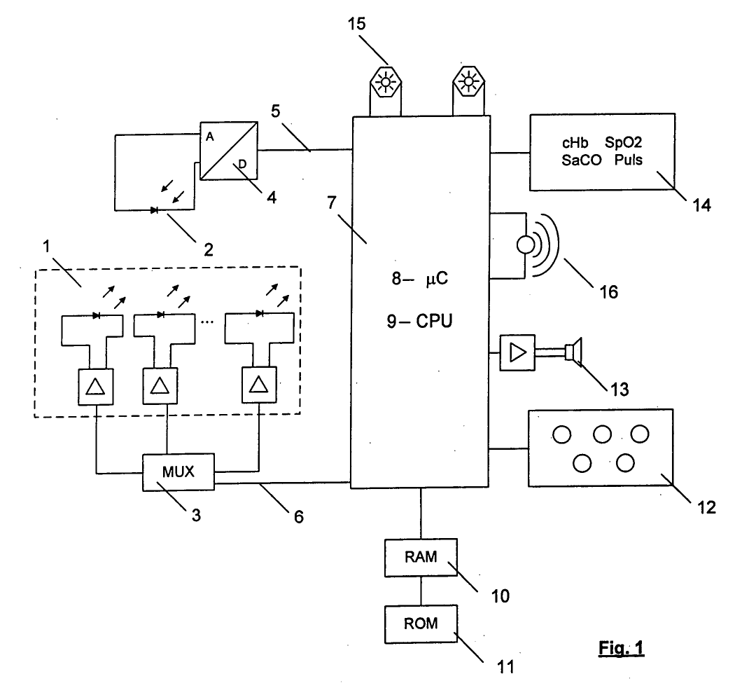

An inventive signal transmitter according to FIG. 1 has a transmitter 1, in which at least one light-emitting diode LEDₐ with a first predetermined nominal wavelength λₐ is located.

Opposite the transmitter unit is a photodetector PD 2. Between the transmitter unit 1 and the photodetector PD 2, human and/or tissue and/or vessel can be placed in such a way that the light emitted by the transmitter unit 1 passes through the tissue and/or the vessel and reaches the photodetector PD 2. The intensity of the light received by the PD is converted to an electrical variable and processed as an analog signal in the device, converted in an A/D converter, and subjected to further digital processing.

The light-emitting diodes LEDₐ, LEDₙ are connected to a multiplexer MUX 3. The control unit of the multiplexer MUX 3 controls the light-emitting diodes so that, in the case that four LEDs are connected, for example, the four LEDs are turned on and off in alternation.

The multiplexer MUX 3 has another terminal 6, which is connected to the evaluation unit 7. By means of this connection with the evaluation unit 7, the data pertaining to the power-on times of the light-emitting diodes LEDₐ-LEDₙ are transmitted. The evaluation unit has at least one microcontroller 8 or at least one CPU 9.

The output current of the photodetector PD 2 is sent to the input of a current/voltage converter 4. The current/-voltage converter 4 converts the output current of the photodetector to an output voltage. In addition, the analog signal of the PD is digitized by an A/D converter of at least 8 bits and transmitted by way of an actuator to the evaluation unit 7. At least one volatile memory unit, namely, RAM 10, and a nonvolatile memory ROM (11) are connected to the evaluation unit 7. The nonvolatile memory 11 is in the form of, for example, an EEPROM or flash memory. An algorithm which serves to determine the pV is stored in the nonvolatile memory 11. An input device 12 in the form of a keyboard can be connected to the evaluation unit 7. In addition, various output devices 13, 14, 15 can also be connected to the evaluation unit 7. By means of a loudspeaker 13, warning tones or voice output can be generated, for example, to inform the user or give him directions. By means of indicator lamps 14, warning signals and/or status signals can be generated. The measurement values are displayed on a display 14.

In at least one operating mode of the inventive device according to FIG. 1 shown by way of example, the tissue/vessel is exposed alternately to the light emitted by the first light-emitting diode LEDₐ and then to the light emitted by the other diodes LEDₙ, where the light passing through the tissue/vessel is received by the photodetector PD and converted to a photodetector output current. The light-emitting diodes LEDₐ, LEDₙ can be operated in binary fashion, which means that at any one point the LED is either emitting light at a predetermined wavelength or not emitting any light at all. Alternatively, the LED can be driven by an analog signal of predetermined amplitude. The timing at which the LED is driven can be a function of the pulse wave phases, such as, for example, every 200 μsec.

The LEDs can be driven as follows at the two times t₁ and t₂:

| | t₁ | t₂ |
|---|---|---|
| 1. | wavelength a | wavelength a |
| 2. | wavelength b | wavelength b |
| 3. | wavelength c | wavelength c |
| 4. | wavelength d | wavelength d |
| 5. | dark | dark |

To convert the current signal with as little noise as possible and with sufficient amplification into a voltage signal which can be used for further processing in the evaluation unit 7, it is sent to the current/voltage converter 4 and to the A/D converter. On the basis of the voltage signal, the evaluation unit 7 determines the change over time in the spectral absorption of the tissue/vessel at the LED-defined wavelengths of the first and/or additional light-emitting diodes LEDₐ, LEDₙ, and by subjecting these spectral absorption values to processing and/or further processing and/or linking, it determines the measurement value of interest at the moment in question, such as the absolute or relative hemoglobin concentration cHb, the COHb concentration, the arterial oxygen saturation SaO₂, CaO₂, or the heart rate. The measurement values for the pV for each wavelength are stored in volatile 10 and/or nonvolatile 11 memory. Then the measurement values are read out again by the evaluation unit 7 with the help of the microcontroller 8 and analyzed in the CPU 9 by means of the algorithm stored in ROM 11.

Digitized data which represent the attenuation and/or scattering of electromagnetic radiation by the tissue/vessel are processed in the central unit under program control, where a control unit retrieves the program commands from a memory unit and uses an ALU, which consists of at least one arithmetical and logical unit, to execute the operations according to the program's instructions.

As a result, absolute and/or relative measurement values are obtained for the desired pV. As a function of, for example, limit values or presettings which can be defined by input on a keyboard 12, for example, the measurement value results are made available as output either electronically, visually 14, 15, and/or acoustically 13. For this purpose, the data which represent the pV are conditioned for an interface and made available to an interface. A protocol is preferably made available via an interface. For example, a voltage and/or a current which is essentially proportional to the pV is made available at the interface. Thus a digitized value representing the pV can be made available in a TCP/IP protocol over an Ethernet connection.

For example, the measurement value can be made available via a proprietary protocol at a UART interface.

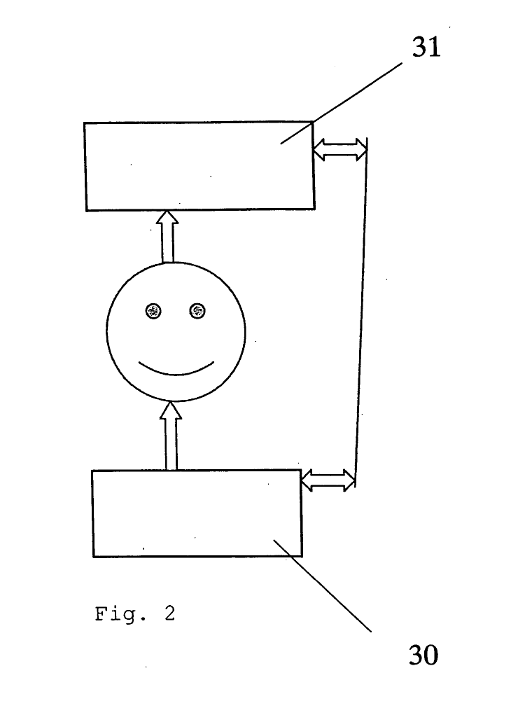

FIG. 2 shows a control circuit consisting of a signal transmitter 30 which emits electromagnetic waves, especially light, of at least two different wavelengths and/or of at least two different wavelength bands from at least one source, where the electromagnetic waves are conducted through human tissue and/or vessels to be examined, and the transmitted and/or reflected component of the electromagnetic waves are detected by a receiver system 31. The receiver system is able to detect the light signals of various wavelengths within a time interval of less than a second, to convert them into current and/or voltage signals which correspond to at least one measurement value, and to transmit them, where at least one measurement value is processed by an evaluation unit by means of a process of signal conditioning and where, independently of the original wavelength, a measurement value is subjected to further processing by active and/or passive electronic components so that it can be transmitted over an interface to a signal receiver, where the transmitted measurement value can be used by the signal receiver to influence directly or indirectly the physiological parameters responsible for the measurement value.

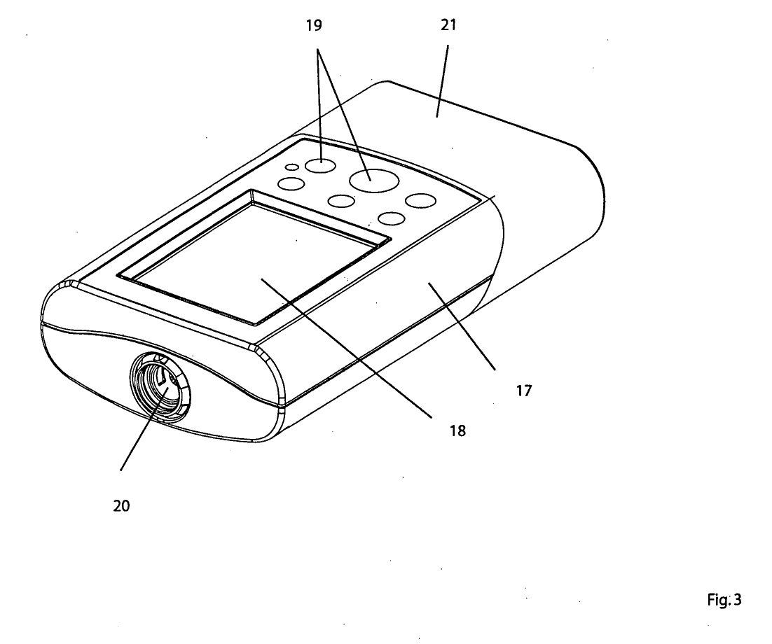

As shown in FIG. 3, another aspect of the invention pertains to a small, portable, handy signal transmitter, which makes it possible for the user to determine several physiological parameters noninvasively. The signal transmitter consists of a housing 17 of plastic with a display 18 and operating buttons 19. The display 18 is connected electrically and mechanically to the main circuit board. An interface 20 is provided in the area of the housing 17. The interface 20 can be connected electrically and mechanically to the main circuit board. The interface serves to accept a sensor cable. Alternatively, the interface can be equipped as a receiving/transmitting module for the wireless transmission of sensor signals to a signal receiver.

In the area of the lower shell 21, there is a socket device for an energy supply such as storage batteries. In the assembled state, the bottom shell 21 and the housing 17 are connected detachably to each other. The dimensions of the inventive signal transmitter are preferably less than 15 cm in length and less than 5 cm in depth and less than 8 cm in width. The volume of the signal transmitter is preferably less than 600 ccm. To achieve small, compact dimensions and nevertheless to ensure that the device can be easily taken apart and reassembled, the signal transmitter consists of no more than two circuit boards and/or fewer than 11 individual parts and/or fewer than three fastening devices.

In an exemplary embodiment, an analog or digital signal is generated from the pV, and this signal is made available over an interface to internal and/or external signal receivers so that the pV itself can be controlled in the physiological sense either manually or automatically. Through feedback and comparison with the actuating variable, a signal for controlling the pV can be determined, as a result of which an automatic control circuit extending across the patient can be constructed. It is especially important here that the measurement signal be evaluated within a defined period of time, such as within a period of seconds, and made available as a actuating variable to the actuator.

To acquire the pV, the methods described in DE 103 21 338 A1 and DE 102 13 692 A1, for example, can be used. The methods from DE 103 21 338 A1 and DE 102 13 692 A1 are to be understood as a component of this application.

In an alternative exemplary embodiment, a control circuit consists of a signal transmitter, which emits electromagnetic waves, especially light, of at least two different wavelengths and/or of at least two different wavelength bands from at least one source, where the electromagnetic waves are conducted through a living and/or dead medium to be examined, preferably animal and/or human tissue and/or vessels, and the transmitted and/or reflected component of the electromagnetic waves are detected by a receiver system, where the receiver system is able to detect the light signals of various wavelengths, to convert them into current and/or voltage signals which correspond to at least one measurement value, and to transmit them within a time interval of less than a second.

At least one measurement value is processed by an evaluation unit through a process of signal conditioning and where, independently of the original wavelength, a measurement value is subjected to further processing by active and/or passive electronic components so that it can be transmitted over an interface to a signal receiver. The transmitted measurement value can be used by the signal receiver to influence directly or indirectly the physiological parameters responsible for the measurement value.

According to another exemplary embodiment, the inventive signal transmitter is able to transmit measurement values which correspond to at least one pV at the time of the measurement to a ventilator, to which an oxygen supply can be connected. In this example, SaO₂ and/or CaO₂ and/or cHb is determined. Data which represent SaO₂ and/or CaO₂ and/or cHb at the time of the measurement are transmitted via an interface to a connected ventilator. SaO₂ and/or CaO₂ and/or cHb are determined, processed, and transmitted to the ventilator in such a way that data which represent the current measurement values are transmitted with a delay of less than 30 seconds, preferably of less than 15 seconds, and even more preferably of less than 5 seconds between the emission of the first wavelength and the transmission to the ventilator.

If there is a change in the setting parameters of the ventilator such as pressure, flow, frequency, respiratory minute volume, and/or the oxygen supply rate, the data which represent SaO₂ and/or CaO₂ and/or cHb at the time of the measurement can be taken into consideration in such a way that the setting parameters of the ventilator are changed in a direction suitable for improving the oxygen supply to the patient.

According to another embodiment, the inventive signal transmitter is able to transmit measurement values to an infusion pump for administering hematopoietic drugs such as erythropoietin (EPO). In this example, SaO₂ and/or CaO₂ and/or cHb is determined. Data which represent SaO₂ and/or CaO₂ and/or cHb at the time of the measurement are transmitted via an interface to a connected infusion pump. SaO₂ and/or CaO₂ and/or cHb is determined, processed, and transmitted to the infusion pump in such a way that data which represent the current measurement values are transmitted with a delay of less than 5 minutes, preferably of less than 2 minutes, and even more preferably of less than 30 seconds between the emission of the first wavelength and the transmission to the infusion pump.

If there is a change in the setting parameters of the infusion pump, the data which represent SaO₂ and/or CaO₂ and/or cHb at the time of the measurement can be taken into consideration in such a way as to change the administration of EPO in a direction suitable for contributing to a definable, optimal supply of oxygen to the patient under consideration of a definable, tolerated elevation in cHb or in the hematocrit.

According to another embodiment, the inventive signal transmitter is able to transmit measurement values to a calculation unit for determining the dosage of a hematopoietic drug. In this example, SaO₂ and/or CaO₂ and/or cHb is determined. Data which represent SaO₂ and/or CaO₂ and/or cHb at the time of the measurement are transmitted via an interface to a calculation unit. SaO₂ and/or CaO₂ and/or cHb is determined, processed, and transmitted to the calculation unit in such a way that data which represent the current measurement values are transmitted with a delay of less than 5 minutes, preferably of less than 2 minutes, and even more preferably of less than 30 seconds between the emission of the first wavelength and the transmission to the calculation unit.

If there is a change in the setting parameters of the calculation unit, the data which represent SaO₂ and/or CaO₂ and/or cHb at the time of the measurement can be taken into consideration in such a way that the calculation unit changes the recommendation for the dosage of a hematopoietic drug in a direction suitable for contributing to a definable, optimal supply of oxygen to the patient under consideration of a definable, tolerated elevation in cHb or in the hematocrit or blood volume.

According to the invention, it is possible for the first time to determine noninvasively physiological parameters such as the parameters of oxygen supply in the periphery of the body by means of a signal transmitter and to provide this information in such a way that, as a function of the determined measurement values, the oxygen supply can be influenced directly and/or indirectly by a signal receiver within an interval of less than 5 minutes, preferably of less than 2 minutes, and even more preferably of less than 30 seconds.

According to another exemplary embodiment, the inventive signal transmitter is able to transmit measurement values to a dialysis machine. In this example, SaO₂ and/or CaO₂ and/or cHb is determined. Data which represent SaO₂ and/or CaO₂ and/or cHb at the time of the measurement are transmitted via an interface to a connected dialysis machine. SaO₂ and/or CaO2 and/or cHb are determined, processed, and transmitted to the dialysis machine in such a way that data which represent the current measurement values are transmitted with a delay of less than 5 minutes, preferably of less than 2 minutes, and even more preferably of less than 30 seconds between the emission of the first wavelength and the transmission to the dialysis machine.

If there is a change in the setting parameters of the dialysis machine, the data which represent SaO₂ and/or CaO₂ and/or cHb at the time of the measurement can be taken into consideration in such a way that the hemofiltration time is changed in a direction suitable for contributing to a definable, optimal supply to the patient under consideration of a definable, tolerated elevation/lowering of cHb or hematocrit.

While specific embodiments of the invention have been shown and described in detail to illustrate the inventive principles, it will be understood that the invention may be embodied otherwise without departing from such principles.

1. A signal transmitter comprising a device for processing, using, and/or providing signals which are generated from measurement values of a physiological variable, wherein
a sequence control approach is implemented in such a way that the measurement values are acquired from the detection of electromagnetic waves of various wavelengths, which have previously passed through a medium to be examined, especially tissue and/or vessels of a living or dead organism, and/or have been reflected by this medium; wherein
at least for a certain percentage of the overall signal quantity, at least two measurement values are used for each generated signal, these values being so close together in time that they form a common signal; and wherein
the signals are intended to be transmitted to a signal receiver, which can be used to support the therapy and/or treatment of an organism.

2. A signal transmitter according to claim 1, wherein, during the generation of the signals, a control device assigned to the signal transmitter takes into account the possible effects of feedback in a control circuit extending from the signal transmitter to the signal receiver and across the organism back to the signal transmitter.

3. A signal transmitter according to claim 1, wherein not only the measurement values but also parameters predefined by the signal receiver are used to generate the signals.

4. A signal transmitter according to claim 1, wherein the signal quality of the signal transmitter is checked for reliability by the signal transmitter itself on the basis of predefined and/or predefinable data, which are stored retrievably within the range of the signal transmitter and/or signal receiver.

5. A signal transmitter according to claim 1, wherein the degree of signal reliability of the signals of the signal transmitter is adjusted in accordance with parameters of the signal receiver being transmitted.

6. A signal transmitter according to claim 1, wherein not only the measurement values but also parameters transmitted by the signal receiver are determined for the signal quantity.

7. A signal transmitter according to claim 1, wherein not only the measurement values but also parameters predefined by the signal receiver are used for the signal quality of the signals.

8. A signal transmitter according to claim 1, wherein the type and/or scope of signal generation by the signal transmitter is also adjusted according to the parameters of the signal receiver being transmitted at the time in question.

9. A signal transmitter according to claim 1, wherein, during the generation of the signals, a control unit assigned to the signal transmitter takes into account the possible effects of feedback in a control circuit extending from the signal transmitter to the signal receiver and back to the signal transmitter via the organism, where the degree to which the signals of the signal transmitter can be controlled is also adjusted according to the parameters of the signal receiver being transmitted at the time in question.

10. A signal transmitter according to claim 1, wherein the degree of the feedback quality of the signals of the signal transmitter is also adjusted according to the parameters of the signal receiver being transmitted at the time in question.

11. A signal transmitter according to claim 1, wherein the physiological variable is selected from the following group:
the concentration of total hemoglobin cHb, of oxyhemoglobin HbO₂, of deoxygenated hemoglobin HbDe, of carboxyhemoglobin HbCO, of methemoglobin HbMet, of sulfhemoglobin HbSulf, of bilirubin, of bile pigments or the absolute oxygen saturation SaO₂, the relative oxygen saturation SpO₂, the oxygen supply CaO₂, or the carbon monoxide saturation SaCO.

12. A signal transmitter according to claim 1, wherein the measurement value of at least one pV is determined within 10 seconds after registration of the detected electromagnetic waves.

13. A signal transmitter according to claim 1, wherein the measurement value is determined and the signal made available for transmission via an interface within one minute after recording the detected electromagnetic waves.

14. A signal transmitter according to claim 1, wherein the measurement values of the saturation SaCO of the exposed medium correspond to the actual saturation with an accuracy of at least 2% in the range of 0-40% saturation.

15. A signal transmitter according to claim 1, wherein the measurement values of the saturation SaO₂ of the exposed medium correspond to the actual saturation with an accuracy of at least 2% in the range of 0-100% saturation.

16. A signal transmitter according to claim 1, wherein the measurement values of the saturation of methemoglobin of the exposed medium correspond to the actual saturation with an accuracy of at least 2% in the range of 0-80% saturation.

17. A signal transmitter according to claim 1, wherein the measurement values of the concentration of hemoglobin of the exposed medium correspond to the actual concentration with an accuracy of at least 2.0 g/dL.

18. A signal transmitter according to claim 1, wherein, from the time at which the electromagnetic waves pass through the tissue/vessel until the output of a value representing cHb, no more than 60 seconds elapse, where the concentration of cHb of the exposed tissue/vessel is accurate to at least 3 g/dL.

19. A signal transmitter according to claim 1, wherein, from the time at which the electromagnetic waves pass through a tissue/vessel until the output of a value representing SaCO, no more than 20 seconds elapse, where the saturation SaCO is accurate to at least 2% (in the measurement range of 0-40%).

20. A control circuit comprising a signal transmitter, which, with a signal receiving system, detects at least one physiological variable of a human being or animal and, with a signal evaluation unit, converts it into processed signals and transmits these signals to a signal receiver, where the transmitted processed signal is used in the signal receiver to influence at least one physiological parameter of the human being or animal with a direct or indirect effect on the physiological variable measured by the signal transmitter.

21. A control circuit according to claim 20, wherein the signal transmitter is a device comprising a sequence control approach is implemented in such a way that the measurement values are acquired from the detection of electromagnetic waves of various wavelengths, which have previously passed through a medium to be examined, especially tissue and/or vessels of a living or dead organism, and/or have been reflected by this medium; wherein
at least for a certain percentage of the overall signal quantity, at least two measurement values are used for each generated signal, these values being so close together in time that they form a common signal; and wherein
the signals are intended to be transmitted to a signal receiver, which can be used to support the therapy and/or treatment of an organism.

22. A control circuit according to claim 21, wherein the signal receiving system of the signal transmitter detects a light signal of a new wavelength after a predefined time interval.

23. A control circuit according to claim 21, wherein the predefined time interval is 1 second.

24. A control circuit according to claim 21, wherein the connection between the signal transmitter and the signal receiver is based on analog and/or digital signals, which are made available via an interface.

25. A control circuit according to claim 21, wherein the connection between the signal transmitter and the signal receiver is achieved through coupling elements for establishing a detachable mechanical and/or electronic connection between the signal transmitter and the signal receiver.

26. A control circuit according to claim 21, wherein the connection between the signal transmitter and the signal receiver can be established by a wireless connection between the signal transmitter and the signal receiver.

27. A control circuit according to claim 21, wherein the signal transmitter and/or the signal receiver has an interface by which it can be connected to additional signal transmitters and/or signal receivers such as analyzers, ECG machines, or sensor devices.

28. A control circuit according to claim 21, wherein data and/or control commands are exchanged between signal transmitters and/or signal receivers.

29. A control circuit according to claim 21, wherein the functionality of the connection between signal transmitters and signal receivers is checked, and a malfunctioning or a properly functioning connection between signal transmitters and signal receivers is communicated to the user via suitable optical and/or acoustic signal elements.

30. A control circuit according to claim 21, wherein the data connection is released through the connection between the signal transmitter and the signal receiver.

31. A control circuit according to claim 21, wherein physiological reactions of the patient which can be caused by the signal receiver are detected by the signal transmitter and sent onward for evaluation and/or remote diagnosis.

32. A control circuit according to claim 21, wherein the signal transmitter identifies different signal receivers on the basis of a code carried by each of the signal receivers.

33. A control circuit according to claim 21, wherein the signal transmitter provides different suitable presettings for each of the individual signal receivers.
